(19) United States
(12) Patent Application Publication
(10) Pub. No.: US 2010/0261739 A1
(43) Pub. Date: Oct. 14, 2010

(54) Method of Treating Non-Small Cell Lung Cancer

(75) Inventor: Mark Laughlin, Sunnyvale, CA (US)

Correspondence Address: Myrexis, Inc.;c/o CPA Global, P.O. Box 52050, Minneapolis, MN, 55402, US

(73) Assignee: Myriad Pharmaceuticals, Inc., Salt Lake City, UT (US)

(21) Appl. No.: 12/706,330

(22) Filed: Feb. 16, 2010

Related U.S. Application Data
(63) Continuation of application No. PCT/US2008/073518, filed on Aug. 18, 2008.
(60) Provisional application No. 60/956,300, filed on Aug. 16, 2007.

(51) Int. Cl. A61K 31/517; A61P 35/00; A61P 35/04

(52) U.S. Cl. 514/266.4

(57) ABSTRACT

A method of treating non-small cell lung cancer (NSCLC), and especially NSCLC that has metastasized to brain is disclosed.

### CROSS REFERENCE TO RELATED U.S. APPLICATION

This application is a continuation of International Application No. PCT/US2008/073518, filed Aug. 18, 2008 and published as WO 2009/023876, which claims the benefit of U.S. Provisional Application Ser. No. 60/956,300, filed on Aug. 16, 2007, both of which are incorporated by reference herein in their entirety.

### FIELD OF THE INVENTION

This invention generally relates to treatment of cancer, and particularly to the use of (4-Methoxy-phenyl)-methyl-(2-methyl-quinazolin-4-yl)-amine to treat non-small cell lung cancer (NSCLC).

### BACKGROUND OF THE INVENTION

Cancer is a common cause of death in the world—about 10 million new cases occur each year, and cancer is responsible for 12% of deaths worldwide, making cancer the third leading cause of death. World Health Organization, National Cancer Control Programmes: Policies and Managerial Guidelines (2d ed. 2002).

NSCLC is a cancer in which malignant cells form in the tissues of the lung. According to the American Cancer Society, there are expected to be approximately 185,000 Americans diagnosed with NSCLC in 2007. Histological examination of NSCLC reveals that NSCLC is a heterogeneous aggregate of histologies, of which the most common histologies are epidermoid or squamous carcinoma, adenocarcinoma, and large cell carcinoma.

Upon diagnosis of NSCLC, patients can be classified into groups that reflect the extent of the disease and treatment approach. Treatment options vary according to such classification, but often include surgery, radiation therapy, and/or administration of chemotherapy drugs.

However, stopping the spread of NSCLC (especially to the brain) has proven to be very diffucult. About 80% of all patients with non-small-cell lung cancer who survive for more than two years, will have brain metastases. Unfortunately, most drugs are not able to cross the blood-brain barrier. Once a tumor of non-small-cell lung cancer origin has been found in the brain, median survival is generally reported as just three to six months. Prior art discloses numerous anti-cancer compounds. For example, PCT Publication No. WO 2005/003100 discloses anti-cancer compounds. However, there are very few drugs approved and effective for treating NSCLC that has metastasized to brain. Thus, there is strong need for compounds that can cross the blood-brain barrier and that are effective in treating NSCLC and NSCLC that has metastasized to the brain.

### SUMMARY OF THE INVENTION

The present invention provides a method of treating or preventing non-small cell lung cancer (NSCLC), especially non-small cell lung cancer (NSCLC) that has metastasized to central nervous system (brain and/or spinal cord) by administering to a patient in need of treatment a therapeutically effective amount of (4-methoxy-phenyl)-methyl-(2-methyl-quinazolin-4-yl)-amine, or a pharmaceutically acceptable salt or solvate thereof.

In one aspect, the present invention provides a method of treating non-small cell lung cancer (NSCLC) comprising administering to a patient in need of treatment a medicament having a therapeutically effective amount of (4-methoxy-phenyl)-methyl-(2-methyl-quinazolin-4-yl)-amine, or a pharmaceutically acceptable salt or solvate thereof.

In another aspect, the invention provides a method of treating a non-small cell lung cancer (NSCLC) patient to delay the onset of metastasis to the CNS. The method comprises administering to a non-small cell lung cancer (NSCLC) patient who does not have CNS (brain or spinal cord) metastasis a medicament having a therapeutically effective amount of (4-methoxy-phenyl)-methyl-(2-methyl-quinazolin-4-yl)-amine, or a pharmaceutically acceptable salt or solvate thereof wherein the metastasis of the tumor into to CNS is delayed or prevented.

In yet another aspect, the invention provides a method of treating non-small cell lung cancer (NSCLC) that has metastasized to the CNS (brain or spinal cord). The method comprises administering to a non-small cell lung cancer (NSCLC) patient who has CNS (brain or spinal cord) metastasis a medicament having a therapeutically effective amount of (4-methoxy-phenyl)-methyl-(2-methyl-quinazolin-4-yl)-amine, or a pharmaceutically acceptable salt or solvate thereof.

It has been discovered that (4-Methoxy-phenyl)-methyl-(2-methyl-quinazolin-4-yl)-amine hydrochloride is tolerated in human patients at certain dosing levels. Accordingly, specific amounts of (4-methoxy-phenyl)-methyl-(2-methyl-quinazolin-4-yl)-amine most suitable for the method of the present invention are provided. In some embodiments, a dose of not more than about 4.5 mg/m², preferably from about 0.3 to about 3.3 mg/m², or between about 2.1 mg/m² and about 3.3 mg/m² of (4-methoxy-phenyl)-methyl-(2-methyl-quinazolin-4-yl)-amine or a molar equivalent amount of a pharmaceutically acceptable salt or solvate thereof, is administered in each administration. In some embodiments, a dose of not more than about 10 mg, preferably between about 0.5 and about 6.6 mg, or between about 4 mg and about 6.6 mg of (4-methoxy-phenyl)-methyl-(2-methyl-quinazolin-4-yl)-amine or a molar equivalent amount of a pharmaceutically acceptable salt or solvate thereof, is administered in each administration.

In various aspects of the present invention and the embodiments thereof, a combination therapy is used. That is, the patient in need of treatment or prevention is additionally treated with another anti-cancer agent, particularly temozolomide (Temodar®). In preferred embodiments, temozolomide is used in the method of treating NSCLC patients who have brain metastasis.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

The present invention provides a method of treating or preventing non-small cell lung cancer (NSCLC), especially non-small cell lung cancer (NSCLC) that has metastasized to central nervous system (brain and/or spinal cord) by administering to a patient in need of treatment a therapeutically effective amount of (4-methoxy-phenyl)-methyl-(2-methyl-quinazolin-4-yl)-amine, or a pharmaceutically acceptable salt or solvate thereof.

It has been discovered that (4-Methoxy-phenyl)-methyl-(2-methyl-quinazolin-4-yl)-amine hydrochloride is safely tolerated in human patients at certain concentrations and dosing levels. Two Phase 1 studies have been performed as open-label, dose-escalating, multiple-dose studies to define the safety, tolerability and phamacokinetics of weekly intravenous administration of (4-Methoxy-phenyl)-methyl-(2-methyl-quinazolin-4-yl)-amine hydrochloride. In particular, (4-Methoxy-phenyl)-methyl-(2-methyl-quinazolin-4-yl)-amine hydrochloride has been administered to patients with NSCLC and to patients with NSCLC that has metastasized to the CNS. The results of such Phase 1 studies show that (4-Methoxy-phenyl)-methyl-(2-methyl-quinazolin-4-yl)-amine hydrochloride is safe and well tolerated in human subjects at selected dosages.

Accordingly, an aspect of the present invention is directed to the use of (4-Methoxy-phenyl)-methyl-(2-methyl-quinazolin-4-yl)-amine, or a pharmaceutically acceptable salt or solvate thereof, such as the hydrochloride salt, as therapy or prophylaxis for NSCLC and/or NSCLC that has metastasized to the brain. In certain embodiments, (4-Methoxy-phenyl)-methyl-(2-methyl-quinazolin-4-yl)-amine, or a pharmaceutically acceptable salt or solvate thereof, such as the hydrochloride salt, is administered at a dose of not more than about 4.5 mg/m² or not more than about 3.3 mg/m².

Dosages are given herein on a hydrochloride salt basis. The dosage of the free base form and different salt and/or solvate forms will vary relative to the differences in molecular weight of those forms to the hydrochloride salt form. One of ordinary skill in the art readily understands how to calculate the molar equivalent of the free base form, or the molar equivalent of any other salt and/or solvate forms. Embodiments of the invention include the appropriate molar equivalents of the dosages given herein.

In some embodiments, (4-Methoxy-phenyl)-methyl-(2-methyl-quinazolin-4-yl)-amine, or a pharmaceutically acceptable salt or solvate thereof, such as the hydrochloride salt, is administered at a dose of not more than about 2.7 mg/m². In further embodiments, (4-Methoxy-phenyl)-methyl-(2-methyl-quinazolin-4-yl)-amine, or a pharmaceutically acceptable salt or solvate thereof, such as the hydrochloride salt, is administered at a dose of not more than about 2.1 mg/m². In particular embodiments, the invention provides a method for treating NSCLC and NSCLC that has metastasized to the brain by administering to an animal (4-Methoxy-phenyl)-methyl-(2-methyl-quinazolin-4-yl)-amine, or a pharmaceutically acceptable salt or solvate thereof, such as the hydrochloride salt, at a dose of between about 0.3 and 3.3 mg/m², such as between about 2.1 mg/m² and about 3.3 mg/m².

The dosage of (4-Methoxy-phenyl)-methyl-(2-methyl-quinazolin-4-yl)-amine, or a pharmaceutically acceptable salt or solvate thereof, such as the hydrochloride salt, may be in mass units (e.g., mg) instead of in mass per Body Surface Area (BSA) (e.g., mg/m²) basis. For example, the dosage may be not more than about 10 mg, about 9 mg, about 8 mg, about 6 mg, about 5 mg, about 4 mg, about 3 mg, about 2 mg, about 1 mg, or about 0.5 mg. Or, in another example, the dosage may be between about 6 mg and about 0.5 mg, such as between about 4 mg and about 6 mg. The conversion from mg/m² to mg may be performed by using an average body surface area (BSA) factor. Exemplary average BSA factors that may be used include 1.7 m² per adult, 1.9 m² per adult male, and 1.7 m² per adult female. Alternatively, the conversion from mg/m² to mg may be performed by using a BSA calculated specifically for a particular subject. Methods of calculating BSA's are known in the art and, thus, are not discussed in more detail herein.

In one embodiment, the invention provides a method of reducing the size or slowing the growth of NSCLC and/or NSCLC that has metastasized to the brain. Reductions in size and/or growth of neoplasms may be measured by the Response Evaluation Criteria in Solid Tumors (RECIST) Guidelines (see Therasse et al. J. Nat. Cancer Institute 92:205-216 (2000), herein incorporated by reference in its entirety). For example, the method may reduce the average size of lesions in patients by about 30% or more as measured at four weeks post-treatment by identifying up to 5 lesions per organ and 10 lesions in total, and determining the reduction in length at the longest diameter of the lesion. In yet another embodiment, the invention provides a method for improving the survival of patients with or at risk of forming brain tumors. The method of the present invention can be applied to all NSCLC subtypes, such as, for example, epidermoid or squamous carcinoma, adenocarcinoma, and large cell carcinoma.

Another aspect of the present invention relates to the administration of (4-Methoxy-phenyl)-methyl-(2-methyl-quinazolin-4-yl)-amine, or a pharmaceutically acceptable salt or solvate thereof, such as the hydrochloride salt, in combination with other known chemotherapeutic agents, such as temozolomide. In some embodiments, (4-Methoxy-phenyl)-methyl-(2-methyl-quinazolin-4-yl)-amine hydrochloride is administered as therapy at a dose of not more than about 4.5 mg/m², such as not more than 3.3 mg/m², or not more than about 2.1 mg/m² in combination with other known chemotherapeutic agents, such as temozolomide. In particular embodiments NSCLC and/or NSCLC that has metastasized to the brain or CNS is treated by administering (4-Methoxy-phenyl)-methyl-(2-methyl-quinazolin-4-yl)-amine hydrochloride at a dose of not more than about 4.5 mg/m², such as not more than 3.3 mg/m², or not more than about 2.1 mg/m² in combination with temozolomide. In other particular embodiments, NSCLC and/or NSCLC that has metastasized to the brain or CNS may be treated by administering (4-Methoxy-phenyl)-methyl-(2-methyl-quinazolin-4-yl)-amine hydrochloride at a dose of not more than about 4.5 mg/m², such as not more than 3.3 mg/m², or not more than about 2.1 mg/m² in combination with temozolomide.

In practicing the methods of the present invention, (4-Methoxy-phenyl)-methyl-(2-methyl-quinazolin-4-yl)-amine, or a pharmaceutically acceptable salt or solvate thereof, such as the hydrochloride salt, may be administered together with at least one known chemotherapeutic agent as part of a unitary pharmaceutical composition. Alternatively, (4-Methoxy-phenyl)-methyl-(2-methyl-quinazolin-4-yl)-amine, or a pharmaceutically acceptable salt or solvate thereof, such as the hydrochloride salt, may be administered apart from at least one known cancer chemotherapeutic agent. In one embodiment, (4-Methoxy-phenyl)-methyl-(2-methyl-quinazolin-4-yl)-amine, or a pharmaceutically acceptable salt or solvate thereof, such as the hydrochloride salt, and at least one known cancer chemotherapeutic agent are administered substantially simultaneously, i.e. the compounds are administered at the same time or one after the other, so long as the compounds reach therapeutic levels in the blood at the same time. In another embodiment, the compound of the invention and at least one known cancer chemotherapeutic agent are administered according to their individual dose schedule, so long as the compounds reach therapeutic levels in the blood.

In particular embodiments, (4-Methoxy-phenyl)-methyl-(2-methyl-quinazolin-4-yl)-amine, or a pharmaceutically acceptable salt or solvate thereof, such as the hydrochloride salt, is administered with temozolomide. Temozolomide may be administered at various dosages, such as a dose of not more than about 75 mg/m² per day or at a dose of not more than about 500, 400, or 250 mg/m² per day. For example, temozolomide may be administered from about 50 mg/m² per day to about 250 mg/m² per day before, after or concurrently with administration of (4-Methoxy-phenyl)-methyl-(2-methyl-quinazolin-4-yl)-amine hydrochloride, or a pharmaceutically acceptable salt or solvate thereof. The particular dose of temozolomide may vary according to the dosing schedule. For example, temozolomide may be administered in a dosing schedule of about 75 mg/m² per day, for six weeks, with a two week interval before beginning the dosing schedule again. Alternatively, temozolomide may be administered in a dosing schedule of about 250 mg/m² per day, for five days, with a one month interval before beginning the dosing schedule again. In specific embodiments, NSCLC and/or NSCLC that has metastasized to the brain or CNS is treated with (4-Methoxy-phenyl)-methyl-(2-methyl-quinazolin-4-yl)-amine, or a pharmaceutically acceptable salt or solvate thereof, such as the hydrochloride salt, at a dose of not more than about 4.5 mg/m², such as not more than 3.3 mg/m², or not more than about 2.1 mg/m² in combination with temozolomide administered at a dose of not more than about 500, 400, or 250 mg/m² per day. Variations of such dosing schedules may also before performed in the administration of temozolomide to a patient.

In practicing the present invention, (4-Methoxy-phenyl)-methyl-(2-methyl-quinazolin-4-yl)-amine, or a pharmaceutically acceptable salt or solvate thereof, such as the hydrochloride salt, may be prepared using methods known to those skilled in the art. Specifically, (4-Methoxy-phenyl)-methyl-(2-methyl-quinazolin-4-yl)-amine hydrochloride may be prepared using methods known to those skilled in the art. For example, (4-Methoxy-phenyl)-methyl-(2-methyl-quinazolin-4-yl)-amine hydrochloride may be prepared according to International Pat. Publication No. WO 2005/003100, the contents of which are incorporated herein in their entirety, and as illustrated by the exemplary reaction in Scheme 1.

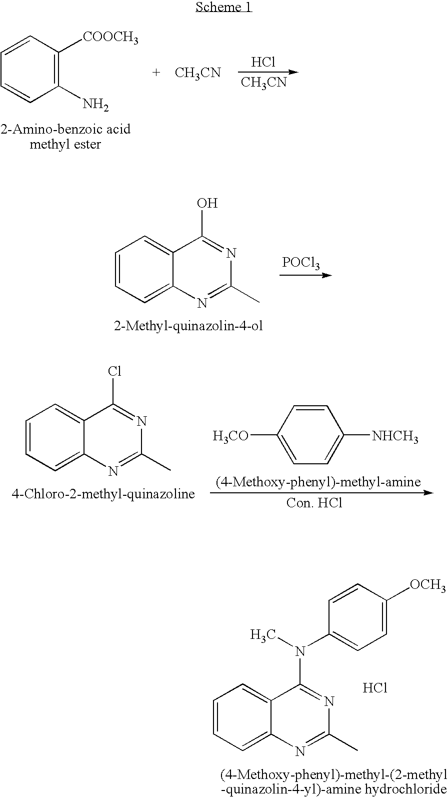

The therapeutic methods of present invention also include methods comprising administering to an animal an effective amount of a compound, or a pharmaceutically acceptable salt, solvate, acid or base of (4-Methoxy-phenyl)-methyl-(2-methyl-quinazolin-4-yl)-amine, such as the hydrochloride salt. In one embodiment, a pharmaceutical composition comprising (4-Methoxy-phenyl)-methyl-(2-methyl-quinazolin-4-yl)-amine, or a pharmaceutically acceptable salt, solvate, acid, or base of said compound, such as the hydrochloride salt, in combination with a pharmaceutically acceptable vehicle is administered. Examples of pharmaceutically acceptable addition salts for (4-Methoxy-phenyl)-methyl-(2-methyl-quinazolin-4-yl)-amine include inorganic and organic acid addition salts, such as hydrochloride, hydrobromide, phosphate, sulphate, citrate, lactate, tartrate, maleate, fumarate, mandelate and oxalate; and inorganic and organic base addition salts with bases, such as sodium hydroxy, Tris(hydroxymethyl)aminomethane (TRIS, tromethane) and N-methyl-glucamine The present invention also includes methods comprising administering to an animal an effective amount of (4-Methoxy-phenyl)-methyl-(2-methyl-quinazolin-4-yl)-amine, or a pharmaceutically acceptable salt thereof, such as the hydrochloride salt, and one or more liquid diluents. Such compositions include compositions disclosed in PCT Pub. No. WO 2006/138608, and may be manufactured according to the methods disclosed therein, the relevant portions of which are incorporated herein by reference in their entirety.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the compounds of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### Example 1

### Preparation of (4-Methoxy-phenyl)-methyl-(2-methyl-quinazolin-4-yl)-amine hydrochloride

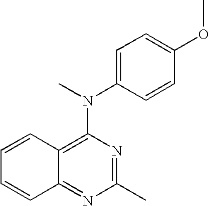

### (4-Methoxy-phenyl)-methyl-(2-methyl-quinazolin-4-yl)-amine hydrochloride

a) 4-Chloro-2-methyl-quinazoline: A stirred suspension of 2-methyl-4(3H)-quinazolinone (5 g, 31.2 mmol) in POCl₃ (100 mL) was heated at 120° C. for 3 h. The excess POCl₃ was removed under vacuum, then to the residue was added crushed ice and 200 mL of saturated NaHCO₃, and the mixture was extracted with ethyl acetate (200 mL×2). The combined extracts were washed with water, saturated NaCl, dried over anhydrous MgSO₄, filtered and concentrated. The crude product was purified by column chromatography (5-8% ethyl acetate/hexane) to give the title compound (2.5 g, 14.0 mmol, 45%). ¹H NMR (CDCl₃): 8.21-8.25 (m, 1H), 7.89-7.99 (m, 2H), 7.66 (ddd, 1H, J=1.8, 6.6, 8.7), 2.87 (s, 3H).

b) (4-Methoxy-phenyl)-methyl-(2-methyl-quinazolin-4-yl)-amine hydrochloride: The title compound was prepared from 4-chloro-2-methyl-quinazoline (2.31 g, 12.9 mmol) and (4-methoxy phenyl)-methyl-amine (2.0 g, 14.6 mmol) by a procedure similar to example 1b and was isolated as solids (2.90 g, 9.18 mmol, 71%). ¹H NMR (CDCl₃): 8.53 (dd, 1H, J=0.6, 8.1), 7.7 (ddd, 1H, J=1.2, 7.2, 8.4), 7.22 (m, 2H), 7.13 (ddd, 1H, J=1.2, 7.2, 8.7), 7.05 (m, 2H), 6.76 (d, 1H, J=8.7), 3.91 (s, 3H), 3.78 (s, 3H), 2.96 (s, 3H).

### Example 2

### Pharmaceutical Composition

A pharmaceutical composition is prepared by combining and mixing 100 grams of (4-Methoxy-phenyl)-methyl-(2-methyl-quinazolin-4-yl)-amine hydrochloride and 1 gram of BHT and dissolving into 10 liters of D5W with the pH adjusted to pH=5 with hydrochloric acid. This solution is sterile filtered using a 0.2 μm Teflon filter (PTFE).

### Example 3

### Pharmaceutical Composition

A pharmaceutical composition was formed by dissolving 300.1 grams (4-Methoxy-phenyl)-methyl-(2-methyl-quinazolin-4-yl)-amine hydrochloride into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 4

### Pharmaceutical Composition

A pharmaceutical composition was formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 13.652 kg viscosity reducing agent (ethanol 190 proof). This solution was sterile filtered through a 0.2 μm Millipore Durapore filter (PVDF), and packaged into 10 ml sterile glass vials.

### Example 5

### Pharmaceutical Composition

A pharmaceutical composition is formed by dissolving 300.1 grams (4-Methoxy-phenyl)-methyl-(2-methyl-quinazolin-4-yl)-amine hydrochloride and 30.12 grams antioxidant (BHT) into 13.652 kg surfactant (CREMOPHOR® EL) and 11.652 kg viscosity reducing agent (ethanol 190 proof), and 2 kg WFI (water for injection). This solution is sterile filtered through a 0.2 μm Millipore Durapore filter (PVDF), and packaged into 10 ml sterile glass vials.

### Example 6

### Method of Administration

About 0.01 ml to about 50 ml of the pharmaceutical composition of Example 5 is accurately measured and then added to an i.v. bag containing about 100 ml to about 1000 ml of sterile dextrose 5% in water (D5W). The amount of pharmaceutical composition and D5W used varies according to the desired therapeutic dose and size of the patient. The resulting mixture is then parenterally infused into the patient.

### Example 7

### Phase I Clinical Trial of Administration of (4-Methoxy-phenyl)-methyl-(2-methyl-quinazolin-4-yl)-amine hydrochloride for Subjects with Refractory Solid Tumors

An open-label, dose-escalating, multiple-dose study to define the safety, tolerability and phamacokinetics of weekly intravenous administration of (4-Methoxy-phenyl)-methyl-(2-methyl-quinazolin-4-yl)-amine hydrochloride was performed. A dosing schedule (each 4 week cycle) was performed for (4-Methoxy-phenyl)-methyl-(2-methyl-quinazolin-4-yl)-amine hydrochloride weekly for 3 weeks with no infusion on the fourth week of each cycle. Subjects with refractory solid tumors were enrolled. During Cycle 1, subjects were hospitalized during each infusion of (4-Methoxy-phenyl)-methyl-(2-methyl-quinazolin-4-yl)-amine hydrochloride and remained for observation and safety evaluation for approximately 24 hours following the end of the infusion. All subject had continuous telemetry for 2 hours prior to infusion, for 1-2 hour infusion and for 3 hours after the end of the infusion. Any clinically significant electrocardiographic (ECG) wave form abnormality was recorded and prolongation of the monitoring period extended at the discretion of the principal investigator.

Electrocardiograms were obtrained prior to starting the infusion and within 30 minutes of the end of infusion for each infusino of the first cycle. Electrocardiograms on Day 1 were obtained in triplicate 5 minutes apart.

Neurocognitive assessments were made by administration of the Mini-Mental State Examination (MMSE), the Hopkins Verbal Learning and timed Grooved Pegboard tests before administration of the intravenous infusion and approximately 24 hours after the infusion at each weekly administration of the first cycle.

On days 1, 8, and 15 of each cycle, vital signs were obtained prior to the first dose, at 15, 30, and 60 minutes after the initiation of the infusion, and at 0.5, 1, 1.5, 2, and 4 hours after the end of the intravenous infusion. Vital signs at all time points beyond the start of the intravenous infusion included heart rate, blood pressure and respirations. Temperature was measured at the end of the infusion and 4 hours later.

Individual subjects were allowed to continue on repeated weekly ×3 administrations every 28 days with no dose increase provided there was no unacceptable toxicity or disease progression.

Tumor response was evaluated by response evaluation criteria in solid tumors

(RECIST) criteria. To prevent sever hypersensitivity reactions due to Cremophor® EL, subjects were premedicated with oral dexamethasone (20 mg) administered approximately 12 and 6 hours before the intravenous infusion with (4-Methoxy-phenyl)-methyl-(2-methyl-quinazolin-4-yl)-amine hydrochloride, diphenhydramine (50 mg) or its equivalent administered intravenously 30-60 minutes before (4-Methoxy-phenyl)-methyl-(2-methyl-quinazolin-4-yl)-amine hydrochloride, and cimetidine (300 mg) or ranitidine (50 mg) administered intravenously 30-60 minutes before (4-Methoxy-phenyl)-methyl-(2-methyl-quinazolin-4-yl)-amine hydrochloride.

Dose escalation of subjects proceeded sequentially as presented in Table 1 below:

TABLE 1

| Cohort | Dose level |
|---|---|
| Number | (modified Fibonacci series) |
| Cohort 1 | Dose = 0.3 mg/m² |
| Cohort 2 | Dose = 0.6 mg/m² |
| Cohort 3 | Dose = 1.0 mg/m² |
| Cohort 4 | Dose = 1.5 mg/m² |
| Cohort 5 | Dose = 2.1 mg/m² |
| Cohort 6 | Dose = 2.7 mg/m² |
| Cohort 7 | Dose = 3.3 mg/m² |
| Cohort 8 | Dose = 3.9 mg/m² |
| Cohort 9 | Dose = 4.5 mg/m² |
| Cohort 10 | Dose = 3.3 mg/m² |

Each cohort included a number of patients. Six patients with NSCLC were part of the study. One patient in Cohort 10 with NSCLC that had metastasized to the CNS continued with the study and was not shown to have disease progression.

There is no evidence from the results of the clinical trials of cytotoxicity peripherally at the administered doses. Additionally, there is no apparent effect of any laboratory parameter was observed, including no reduction neutrophils, platelets, or hemoglobin. There were incidences of intratumor bleeding and the dose limiting toxicity was demonstrated to be vascular in nature, manifested by an acute coronary syndrome, particularly in Cohorts 8 and 9. The optimal dosage derived from the trials is determined to be 3.3 mg/m² or lower. Within this dosage range, (4-Methoxy-phenyl)-methyl-(2-methyl-quinazolin-4-yl)-amine hydrochloride was shown to be safe and tolerated.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of treating non-small cell lung cancer in a mammal in need of such treatment, comprising administering to the mammal an effective amount of (4-Methoxy-phenyl)-methyl-(2-methyl-quinazolin-4-yl)-amine, or a pharmaceutically acceptable salt thereof.

2. The method of claim 1, wherein the non-small cell lung cancer has metastasized to the brain and/or central nervous system.

3. The method of claim 1, wherein the effective amount of (4-Methoxy-phenyl)-methyl-(2-methyl-quinazolin-4-yl)-amine, or a pharmaceutically acceptable salt thereof, is administered at a dose of not more than about 4.5 mg/m², or molar equivalent thereof.

4. The method of claim 1, wherein the effective amount of (4-Methoxy-phenyl)-methyl-(2-methyl-quinazolin-4-yl)-amine, or a pharmaceutically acceptable salt thereof, is administered at a dose of not more than about 3.3 mg/m², or molar equivalent thereof.

5. The method of claim 1, wherein the effective amount of (4-Methoxy-phenyl)-methyl-(2-methyl-quinazolin-4-yl)-amine, or a pharmaceutically acceptable salt thereof, is administered at a dose of not more than about 2.7 mg/m², or molar equivalent thereof.

6. The method of claim 1, wherein the effective amount of (4-Methoxy-phenyl)-methyl-(2-methyl-quinazolin-4-yl)-amine, or a pharmaceutically acceptable salt thereof, is administered at a dose of not more than about 2.1 mg/m², or molar equivalent thereof.

7. The method of claim 1, wherein the effective amount of (4-Methoxy-phenyl)-methyl-(2-methyl-quinazolin-4-yl)-amine, or a pharmaceutically acceptable salt thereof, is administered at a dose between about 3.3 mg/m² and about 0.3 mg/m², or molar equivalent thereof.

8. The method of claim 1, wherein the effective amount of (4-Methoxy-phenyl)-methyl-(2-methyl-quinazolin-4-yl)-amine, or a pharmaceutically acceptable salt thereof, is administered at a dose between about 3.3 mg/m² and about 2.1 mg/m², or molar equivalent thereof.

9. The method of claim 1, wherein the mammal is also administered an effective amount of another chemotherapeutic agent.

10. The method of claim 9, wherein the another chemotherapeutic agent is temozolomide.

11. The method of claim 1, wherein the non-small cell lung cancer comprises squamous cell carcinoma.

12. The method of claim 1, wherein the non-small cell lung cancer comprises adenocarcinoma.

13. The method of claim 1, wherein the non-small cell lung cancer comprises large cell carcinoma.

14. A method of reducing the size or slowing the growth of non-small cell lung cancer in an individual, said method comprising administering to the individual an effective amount of (4-Methoxy-phenyl)-methyl-(2-methyl-quinazolin-4-yl)-amine, or a pharmaceutically acceptable salt thereof.

15. The method of claim 14, wherein said non-small cell lung cancer has metastasized to the brain.

16. The method of claim 14, wherein the pharmaceutically acceptable salt is (4-Methoxy-phenyl)-methyl-(2-methyl-quinazolin-4-yl)-amine hydrochloride.

17. The method of claim 14, wherein said effective amount of (4-Methoxy-phenyl)-methyl-(2-methyl-quinazolin-4-yl)-amine, or a pharmaceutically acceptable salt thereof, is administered at a dose of not more than about 4.5 mg/m², or molar equivalent thereof.

18. The method of claim 14, wherein said effective amount of (4-Methoxy-phenyl)-methyl-(2-methyl-quinazolin-4-yl)-amine, or a pharmaceutically acceptable salt thereof, is administered at a dose of not more than about 3.3 mg/m², or molar equivalent thereof.

19. A method of treating non-small cell lung cancer that has metastasized to the brain in a mammal in need of such treatment, comprising administering to the mammal an effective amount of (4-Methoxy-phenyl)-methyl-(2-methyl-quinazolin-4-yl)-amine or a pharmaceutically acceptable salt thereof.

20. The method of claim 19, wherein an effective amount of (4-Methoxy-phenyl)-methyl-(2-methyl-quinazolin-4-yl)-amine hydrochloride is administered at a dose of not more than about 4.5 mg/m².
